(19) United States
(12) Patent Application Publication
(10) Pub. No.: US 2024/0335375 A1
(43) Pub. Date: Oct. 10, 2024

(54) MUCOADHESIVE OCULAR DELIVERY SYSTEM FOR THE TREATMENT OF GLAUCOMA

(71) Applicants: BIOADHESIVE OPHTHALMICS, Orsay (FR); UNIVERSITÉ DE LORRAINE, NANCY (FR)

(72) Inventors: Jean CUINE, Orsay (FR); Boris BIZET, Orsay (FR); Frédéric LALLEMAND, Orsay (FR); Jean GARREC, Orsay (FR); Stéphane GIBAUD, NANCY (FR); Anne SAPIN-MINET, NANCY (FR)

(73) Assignees: BIOADHESIVE OPHTHALMICS, Orsay (FR); UNIVERSITÉ DE LORRAINE, Nancy (FR)

(21) Appl. No.: 18/294,739

(22) PCT Filed: Aug. 3, 2022
(86) PCT No.: PCT/EP2022/071870

§ 371 Date: Feb. 2, 2024

(30) Foreign Application Priority Data
Aug. 3, 2021 (EP) 21306084.1

(51) Int. Cl. A61K 9/00; A61K 45/06; A61K 47/36

(52) U.S. Cl. CPC A61K 9/0048; A61K 45/06; A61K 47/36

(57) ABSTRACT

An ocular delivery system useful for the treatment of glaucoma in subject in need thereof. Especially, a mucoadhesive solid or semisolid ocular delivery system that includes a matrix of preactivated thiomer of hyaluronic acid and one or more anti-glaucoma drugs. The ocular delivery system may be an ocular insert or an ocular film.

### FIELD OF INVENTION

The present invention relates to the field of ophthalmic formulations and relates to an ocular delivery system useful for the treatment of glaucoma. Especially, the invention provides a mucoadhesive ocular insert comprising a matrix of preactivated thiomer of hyaluronic acid and one or more anti-glaucoma drug.

### BACKGROUND OF INVENTION

Glaucoma is a group of eye conditions that damage the optic nerve and may cause vision loss. This damage is often caused by an abnormally high pressure in the eye. The most common type is open-angle glaucoma, which develops slowly over time, with first a decrease of peripheral vision, followed by central vision loss, resulting in blindness if not treated. Vision loss due to glaucoma, once it has occurred, is permanent. If treated early, it is possible to slow or stop the progression of disease. Treatment of glaucoma can be achieved with medication, laser treatment, or surgery.

Glaucoma medication aims at preserving visual function by lowering intraocular pressure (IOP) below a level that is likely to produce further damage to the nerve. Different classes of IOP lowering agents can be used, such as prostaglandin analogs, cholinomimetics, beta blockers, alpha adrenergic agonists, carbonic anhydrase inhibitors, Rho kinase inhibitors and NO donor agents. Anti-glaucoma drugs are usually administered under the form of eye drops and need to be administered for the rest of the patient's life.

One of the major issues in glaucoma medication is thus patient compliance due to the necessary daily instillation of eye drops. Moreover, the presence of preservatives in the compositions may be irritative and may lead to dry eye.

Another problem of topical ocular administration is to obtain and maintain a sufficient amount of active substance at the site of action for a prolonged period of time, and to deliver a precise dose of active substance. Aqueous eye drops present the drawback to have a rapid draining after instillation, resulting in poor bioavailability. Moreover, several anti-glaucoma drugs are hydrophobic molecules which have poor solubility in water.

Various alternatives to eye drops are available in order to optimize ocular medication, such as for example using emulsions, in situ gelling polymers, microspheres, nanoparticles, liposomes or ocular inserts.

Ocular inserts are solid or semisolid ocular delivery devices to be placed in the conjunctival cul-de-sac of the eye. Ocular inserts offer an interesting alternative to eye drops since they ensure a longer pre-corneal residence time and reduce the amount of systemic absorption. For example, US2015/157563 discloses ocular inserts comprising a polymer matrix, an antimicrobial dispersed therein, and optionally other active substances, for the controlled release of the actives. The polymer matrix contains a thiolated hyaluronic acid moiety (such as a thiolated carboxymethyl hyaluronic acid (CMHA-S) moiety), cross-linked to a second moiety which is preferably poly(ethylene glycol) diacrylate moiety (PEGDA). Additional ingredients are added to provide mucoadhesiveness, such as methylcellulose (MC).

Nevertheless, ocular inserts most often have poor patient acceptance since they lead to a sensation of foreign body in the eye. Further, in the event that the insert moves around the eye, it may also interfere with vision and cause irritation, and it may even lead to its ejection from the conjunctival cul-de-sac, limiting the remanence time. Another drawback of currently available inserts is that their dissolution curves may be too short to enable the release of the drug over few days.

Efforts are thus undertaken to provide mucoadhesive ocular inserts which are well tolerated by patients and remain simple to produce with a release rate of several days.

When an ocular delivery system is placed at the surface of the eye, it is first in contact with the tear film, which is formed of three layers: lipid layer, aqueous layer and mucin layer. The mucins present under the tear film can thus be targeted in order to obtain the adhesion of the ocular delivery system to the eye surface. Several polymers were already tested for their mucoadhesive properties, such as thiomers.

Thiomers, also referred to as “thiolated polymers”, are polymers having side chains bearing free thiol moieties (Bernkop-Schnürch A. et al., Pharm. Res., 1999, 16, 876-881; U.S. Pat. No. 7,354,600; Bonengel S. and Bernkop-Schnürch A., J. Controlled Release, 2014, 120-129). The polymeric backbone of thiomers usually consists of biodegradable polymers, such as for example chitosan, hyaluronic acid, gelatin, polyacrylates, cellulose derivatives, cyclodextrins or silicones. The thiolation of such polymeric backbones may be performed for example by coupling cysteine moieties. Thiomers are capable of forming covalent bonds, namely disulfide bonds, with cysteine-rich subdomains of mucins covering mucosal membranes. Such covalent bonds are strong and thus enable to ensure an efficient mucoadhesion of dosage forms comprising thiomers for a prolonged time.

Hornof et al. tested a mucoadhesive ocular insert based on thiolated poly(acrylic acid), for the controlled release of ophthalmic drugs (Hornof et al., J. Controlled Release, 2003, 419-428). The dry ocular insert is placed in the conjunctival cul-de-sac of the eye and hydrates in situ to form a hydrogel that presents a good mucoadhesion. The hydrogel form does not lead to a foreign body sensation, contrary to previous ocular inserts and mucoadhesion allows the insert to stay in place. Nevertheless, such thiomer ocular inserts must remain stored at a non-physiological pH (for the thiomer ocular insert of Hornof et al., at pH 5) in order to avoid the oxidation of the thiol groups of the thiomer and maintain them under reduced form. This is essential in order to keep a sufficient amount of free thiol groups available for interaction with mucins. Therefore, the main drawback of thiomer ocular inserts is that they provoke irritation and pain due to their non-physiological pH. Moreover, such pH may not be suitable to carry some active substances which are not stable under such conditions. Last, Hornof et al. did not evidence a drug release beyond 8 hours with tested inserts.

There is thus a need for new solid or semisolid ocular delivery systems (including ocular inserts and ocular films) which have effective mucoadhesive properties, are well tolerated by patients and are suitable for the delivery of anti-glaucoma drugs over several days, preferably with a release rate of more than 2 days, more preferably of more than 3 days.

For that purpose, the Applicant herein provides mucoadhesive solid or semisolid ocular delivery systems based on a matrix of preactivated thiomers of hyaluronic acid for the delivery of anti-glaucoma drugs.

Preactivated thiomers, or S-protected thiomers, are thiomers in which the thiol moieties of the side chains are conjugated in disulfide bonds with vitamin B derivatives, such as mercaptonicotinic acids, mercapto (iso) nicotinamides or mercaptopyridoxines (US 2012/0225024). Mucoadhesive properties of such preactivated thiomers were reported, for example with poly(acrylic acid)-cysteine-2-mercaptonicotinic acid (Iqbal J. et al., Biomaterials, 2012, 33, 1528-1535) and for vaginal delivery with hyaluronic acid-L-cysteine-6-mercaptonicotinamide (Nowak J., Int. J. Pharmaceutics, 2015, 478, 383-389).

The Applicant provided, for the first time in WO2021/156435, a mucoadhesive solid or semisolid ocular delivery system comprising a matrix made of at least one preactivated thiomer. Nevertheless, to the knowledge of the Applicant, the use of preactivated thiomers of hyaluronic acid, was never reported before for the manufacturing of ocular delivery systems, such as ocular inserts or ocular films, for the delivery of anti-glaucoma drugs.

The use of hyaluronic acid as backbone of the preactivated thiomer used in the ocular delivery system of the invention is advantageous in terms of ophthalmic tolerance, hydrating and lubricating properties, as gelation properties.

The presence of preactivated thiol groups in the thiomers of hyaluronic acid used in ocular delivery systems of the invention enhances the stability, the mucoadhesion, and the tolerance of the thiomers, and thus provides ocular delivery systems with expected properties. The delivery systems of the invention especially present the advantage to prolong the residence time of the delivery system at the site of application without causing irritation. The adherence to the treatment by patients is improved when using the delivery system of the invention, compared with the use of eye drops, since it avoids repeated instillations and thereby improves patient compliance. Moreover, the therapeutic performance of the delivered anti-glaucoma drugs is improved by increasing their bioavailability. Especially, the ocular delivery systems of the invention enable to increase the residence time of the drugs, which in turn enhances the diffusion of the drugs to the eye. In addition, the thiomers used in ocular delivery systems of the invention reversibly open the tight junctions of the epithelium which in turn enhances the permeation of the drugs. It also enables their sustained release overtime and allows the delivery of more precise doses, compared to what can be achieved when using eye drops.

To be suitable for ocular use, the solid or semisolid delivery systems of the present invention should encounter several specifications, such as having a shape and a size adapted to ocular placement, and enabling a suitable hydration with a controlled swelling. It is also essential that the ocular delivery systems of the invention be adapted and resistant to the shear movements induced by the natural movements of the eyeball combined with the permanent blinking of the eyelids.

### SUMMARY

This invention thus relates to a mucoadhesive solid or semisolid ocular delivery system comprising:
one or more anti-glaucoma drug; and
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups; and wherein the preactivated thiomer of hyaluronic acid has a molecular weight ranging from 100 kDa to 1200 kDa.

In one embodiment, the ocular delivery system is an ocular insert or an ocular film.

In one embodiment, the side chains of the preactivated thiomer of hyaluronic acid are selected from:
S-(2- or 6-mercaptonicotinic acid)-, S-(2- or 6-mercapto (iso) nicotinamide)- or S-(6-mercaptopyridoxine)-cysteine-disulfides,
S-(2- or 6-mercaptonicotinic acid)-, S-(2- or 6-mercapto (iso) nicotinamide)- or S-(6-mercaptopyridoxine)-homocysteine-disulfides,
S-(2- or 6-mercaptonicotinic acid)-, S-(2- or 6-mercapto (iso) nicotinamide)- or S-(6-mercaptopyridoxine)-cysteamine-disulfides,
S-(2- or 6-mercaptonicotinic acid)-, S-(2- or 6-mercapto (iso) nicotinamide)- or S-(6-mercaptopyridoxine)-N-acetylcysteine-disulfides,
S-(2- or 6-mercaptonicotinic acid)-, S-(2- or 6-mercapto (iso) nicotinamide)- or S-(6-mercaptopyridoxine)-thioglycolic acid-disulfides,
S-(2- or 6-mercaptonicotinic acid)-, S-(2- or 6-mercapto (iso) nicotinamide)- or S-(6-mercaptopyridoxine)-3-thiopropionic acid-disulfides,
S-(2- or 6-mercaptonicotinic acid)-, S-(2- or 6-mercapto (iso) nicotinamide)- or S-(6-mercaptopyridoxine)-4-thiobutanoic acid-disulfides,
S-(2- or 6-mercaptonicotinic acid)-, S-(2- or 6-mercapto (iso) nicotinamide)- or S-(6-mercaptopyridoxine)-mercaptobenzoic acid-disulfides,
S-(2- or 6-mercaptonicotinic acid)-, S-(2- or 6-mercapto (iso) nicotinamide)- or S-(6-mercaptopyridoxine)-mercaptonicotinic acid-disulfides,
S-(2- or 6-mercaptonicotinic acid)-, S-(2- or 6-mercapto (iso) nicotinamide)- or S-(6-mercaptopyridoxine)-glutathione-disulfides, and
S-(2- or 6-mercaptonicotinic acid)-, S-(2- or 6-mercapto (iso) nicotinamide)- or S-(6-mercaptopyridoxine)-mercaptoaniline-disulfides;
   said side chains being independently attached to the hyaluronic acid backbone via amide, or ester bonds.

In one embodiment, the preactivated thiomer of hyaluronic acid comprises from 10 μmol to 1350 μmol of mercaptonicotinic acid, mercaptonicotinamide, mercaptoisonicotinamide or mercaptopyridoxine partial structures per gram polymer; preferably from 100 μmol to 1350 μmol per gram polymer.

In one embodiment, the ocular delivery system further comprises a non-preactivated thiomer, preferably a non-preactivated thiomer of hyaluronic acid. In one embodiment, the non-preactivated thiomer is selected from polymeric compounds having a hyaluronic acid backbone bearing covalently bonded thiolated side chains selected from cysteine, homocysteine, N-acetylcysteine, cysteine ethyl ester, cysteamine, mercaptoaniline, adipic acid dihydrazide thiolated by reaction with iminothiolane, 5,5′-dithiobis(2-nitrobenzoic acid), dithiobis(propanoic dihydrazide), dithiobis(butyric dihydrazide), 3-(2-pyridyldithio) propionyl hydrazide, dithiothreitol, ethylene sulfide, thioglycolic acid, 3-thiopropionic acid, 4-thiobutanoic acid, mercaptobenzoic acid, mercaptonicotinic acid, glutathione, and gamma-thiobutyrolactone; said side chains being independently attached to the hyaluronic acid backbone via amide, ether or ester bonds.

In one embodiment, the preactivated thiomer of hyaluronic acid, and/or when present the non-preactivated thiomer, is crosslinked.

In one embodiment, the anti-glaucoma drug is an intraocular pressure (IOP) lowering agent selected from prostaglandin analogs, cholinomimetics, beta blockers, alpha adrenergic agonists, carbonic anhydrase inhibitors, Rho kinase inhibitors, NO donor agents and combinations thereof. In one embodiment, the anti-glaucoma drug is selected from latanoprost, bimatoprost, travoprost, tafluprost, latanoprostene bunod, pilocarpine, echothiophate, carbachol, timolol, nadolol, carteolol, levobunolol, metipranolol, betaxolol, brimonidine, apraclonidine, dorzolamide, brinzolamide, acetalozamide, methazolamide, and netarsudil. In one embodiment, the ocular delivery system comprises two anti-glaucoma drugs, one being a prostaglandin analog and the other being a beta blocker; preferably bimatoprost and timolol.

In one embodiment, the ocular delivery system further comprises one or more pharmaceutically acceptable excipient. In one embodiment, the excipient is selected from: thickening agents, gelling agents, plasticizers, solubilization agents, stabilizing agents, permeation enhancers, diluents, binding agents, glidants, channeling agents, lubricants and modified release agents. In one embodiment, the excipient is selected from: high molecular weight crosslinked polyacrylic acid polymers, polyvinyl alcohol, polyvinylpyrrolidone (also referred to as povidone), cellulose, microcrystalline cellulose, hydroxypropyl cellulose, hydroxypropyl methylcellulose (also referred to as hypromellose), carboxymethyl cellulose, polyethylene glycol, hyaluronic acid, glycerol, cyclodextrins, glutathione in its reduced form, sorbitol, trehalose, xylitol, mannitol, saccharides and their derivatives, sucrose, lactose, polysaccharides and their derivatives, magnesium stearate, dibasic calcium phosphate, colloidal silicon dioxide, sodium chloride, polyoxyethylene stearates, lauryl sulphate salts, hydrogenated coco monoglycerides, hydrogenated coco monoglycerides diglycerides and hydrogenated coco monoglycerides triglycerides, glyceryl behenate, stearic acid, glyceryl palmitostearate, glyceryl dibehenate, glyceryl distearate, ammonio methacrylate copolymer (Type A), polyvinyl acetate-povidone co-polymer, and polyvinyl caprolactam-polyvinyl acetate-polyethylene glycol graft co-polymer.

In one embodiment, the ocular delivery system comprises:
0.01% to 50% in weight of the total weight of the delivery system (w/w) of one or more anti-glaucoma drug;
5% to 80% w/w of at least one preactivated thiomer of hyaluronic acid;
0% to 89.99% w/w of a non-preactivated thiomer of hyaluronic acid; and
0% to 94.99% w/w of one or more pharmaceutically acceptable excipient.

The invention also provides an ocular delivery system as herein defined, for use in the treatment of glaucoma in a subject in need thereof.

### Definitions

In the present invention, the following terms have the following meanings:

“Administration”, or a variant thereof (e.g. “administering”), means providing the active substance, alone or as part of a pharmaceutically acceptable formulation, to the patient in whom/which the condition, symptom, or disease is to be treated.

“Anti-glaucoma drug” refers to a drug used for the treatment of glaucoma.

“Carbomer” refers to synthetic high-molecular-weight polyacrylic acids cross-linked with allyl sucrose or allyl pentaerythritol. Examples of carbomers include Carbopol 971 and 974 which are polyacrylic acids cross-linked with allyl pentaerythritol and polymerized in ethyl acetate.

“Electrospinning” refers to a process that generates a network of tridimensional polymer nanofibers. Electrospinning uses an electrical charge to draw very fine fibers from a liquid. Methods to perform electrospinning are known by skilled artisan.

“Human” refers to a subject of both genders and at any stage of development (i.e. neonate, infant, juvenile, adolescent, adult).

“Mucoadhesive” refers to the attractive forces between a substance or material and mucus or mucosal membrane. In the context of the present invention, a “mucoadhesive ocular delivery system” is an ocular delivery system that strongly interacts with mucus or mucosal membranes of the eye. In a preferred embodiment, the ocular delivery system of the invention covalently binds to the mucus or mucosal membrane by the formation of disulfide bonds between the thiomer and the natural mucins present therein. This disulfide bonds formation is facilitated by the use of preactivated thiomers.

“Nanofiber” refers to a fiber having an average diameter of less than 5000 nm, preferably less than 1000 nm.

“Ocular delivery system” refers to a delivery system which enables to administer an active pharmaceutical ingredient or a substance of interest, such as for example an anti-glaucoma drug, to a subject via an eye or any part thereof. A “solid or semisolid ocular delivery system” refers to solid or semisolid dosage forms including ocular inserts and ocular films. Especially, “semisolid” refers to a dosage form which may be highly viscous, such as an ocular insert under the form of a hydrogel.

“Ocular film” refers to a solid or semisolid consistency bidimensional film designed to be placed into the conjunctival cul-de-sac or at the conjunctival surface, whose size and shape are especially designed for ophthalmic application. Preferably, ocular films are sterile. The ocular film can be folded to form a tridimensional device, what can be useful for example to facilitate the placement of the film on the eye.

“Ocular insert” refers to a solid or semisolid consistency tridimensional device designed to be placed into the conjunctival cul-de-sac or at the conjunctival surface, whose size and shape are especially designed for ophthalmic application. Preferably, ocular inserts are sterile. Optionally, ocular inserts can be multilayered. Ocular inserts can be under dry or hydrated forms. In the latter case, in the present invention, the ocular insert is preferably under the form of a hydrogel pellet.

“Ocular condition” refers to any condition that affects any area of the eyeball, as well as the eyelids. Examples of ocular conditions include ocular conditions after eye surgery, dry eye symptoms and ocular symptoms due to seasonal allergies.

“Pharmaceutically acceptable” refers to the ingredients of a pharmaceutical formulation which are compatible with each other and not deleterious to the subject to which it is administered.

“Pharmaceutically acceptable excipient” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, pH modifying agents, preserving agents (or preservating agents), antibacterial and antifungal agents, isotonifiers, granulating agents or binders, lubricants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

“Polycarbophil” refers to a synthetic polymer manufactured from the cross-linking of polyacrylic acid with divinyl glycol and a calcium counter-ion.

“Polymeric compound” refers to a polymer. In the sense of the present invention, a polymeric compound may comprise a “polymeric backbone” with “side chains”.

“Subject” refers to a mammal, including humans and animals, preferably a human. In one embodiment, the subject is diagnosed with a disease. In one embodiment, the subject is a “patient”, who/which is awaiting the receipt of, or is receiving, medical care or was/is/will be the subject of a medical procedure or is monitored for the development or progression of a disease. In one embodiment, the subject is a male. In another embodiment, the subject is a female. In one embodiment, the subject is an adult. In another embodiment, the subject is a child.

“Therapeutically effective amount” or “effective amount” or “therapeutically effective dose” refer to the amount or dose of active substance that is aimed at, without causing significant negative or adverse side effects to the subject, (1) delaying or preventing the onset of a disease in the subject; (2) reducing the severity or incidence of a disease; (3) slowing down or stopping the progression, aggravation, or deterioration of one or more symptoms of a disease affecting the subject; (4) bringing about ameliorations of the symptoms of a disease affecting the subject; or (5) curing a disease affecting the subject. A therapeutically effective amount may be administered prior to the onset of a disease for a prophylactic or preventive action. Alternatively, or additionally, a therapeutically effective amount may be administered after initiation of a disease for a therapeutic action.

“Treating” or “treatment” refer to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject is successfully “treated” for a disease or condition if, after receiving a therapeutic amount of a therapeutic agent, the patient shows observable and/or measurable reduction in or absence of one or more of the following: relief to some extent, of one or more of the symptoms associated with the specific disease or condition and improvement in quality of life issues. The above parameters for assessing successful treatment and improvement in the disease are readily measurable by routine procedures familiar to a physician.

“Thiomers” or “non-preactivated thiomers” refers to thiolated polymers, i.e. polymers having side chains bearing free thiol moieties (i.e. “thiolated side chains”) linked to a polymeric backbone. The polymeric backbone can be a biodegradable polymer, such as for example hyaluronic acid. The thiolation of the polymeric backbone may be performed for example by coupling cysteine or cysteamine moieties. A “thiomer of hyaluronic acid” refers to a thiomer in which the polymeric backbone is hyaluronic acid.

“Preactivated thiomers” or “S-protected thiomers” refers to thiomers in which the thiol moieties of the side chains are conjugated through disulfide bonds with vitamin B derivatives, such as mercaptonicotinic acids, mercapto (iso) nicotinamides or mercaptopyridoxines. As detailed hereafter, preactivated thiomers can be obtained either by the coupling of vitamin B derivatives on the free thiols of thiomers through the formation of disulfide bonds, or by the direct coupling on a polymeric backbone of side chains comprising vitamin B derivative-disulfide groups. A “preactivated thiomer of hyaluronic acid” refers to a preactivated thiomer in which the polymeric backbone is hyaluronic acid.

“Thiomer matrix” refers to a matrix mainly made of thiomer (preactivated and/or non-preactivated).

### DETAILED DESCRIPTION

### Ocular Delivery System of Anti-Glaucoma Drugs

This invention thus relates to ocular drug delivery systems useful for the treatment of glaucoma. Especially, the invention provides solid or semisolid delivery systems enabling to efficiently deliver anti-glaucoma drugs at the eye level. The drug delivery systems of the invention may be an ocular insert or an ocular film. The ocular delivery systems of the invention present mucoadhesive properties that enable the delivery systems to remain on the ocular surface for extended periods of time. The mucoadhesive properties are achieved by the presence of at least one preactivated thiomer of hyaluronic acid in the delivery system. Once hydrated, the thiomer matrix of the ocular delivery system of the invention forms a hydrogel that adheres to the eye.

Therefore, the invention provides a mucoadhesive solid or semisolid ocular delivery system comprising one or more anti-glaucoma drug in a matrix of preactivated thiomer of hyaluronic acid.

In one embodiment, the ocular delivery system of the invention comprises:
one or more anti-glaucoma drug; and
at least one preactivated thiomer of hyaluronic acid.

### Anti-Glaucoma Drugs

The ocular drug delivery system of the invention is useful for the treatment of glaucoma by enabling the efficient delivery anti-glaucoma drugs at the eye level. The ocular drug delivery system of the invention comprises at least one anti-glaucoma drug.

In one embodiment, anti-glaucoma drugs are selected from intraocular pressure (IOP) lowering agents. IOP lowering agents can decrease the secretion of aqueous humor and/or increase the elimination of aqueous humor from the eye. Examples of IOP lowering agents include prostaglandin analogs, cholinomimetics, beta blockers, alpha adrenergic agonists, carbonic anhydrase inhibitors, Rho kinase inhibitors and NO donor agents. IOP lowering agents that decrease the secretion of aqueous humor include beta blockers, carbonic anhydrase inhibitors, and alpha adrenergic agonists. IOP lowering agents that increase the elimination of aqueous humor include prostaglandin analogs, cholinomimetics.

Examples of prostaglandin analogs include latanoprost, bimatoprost, travoprost, tafluprost, and latanoprostene bunod. Examples of cholinomimetic include pilocarpine, echothiophate, and carbachol. Examples of beta blockers include timolol, nadolol, carteolol, levobunolol, metipranolol, and betaxolol. Examples of alpha adrenergic agonists include brimonidine and apraclonidine. Examples of carbonic anhydrase inhibitors include dorzolamide, brinzolamide, acetazolamide, and methazolamide. Examples of Rho kinase inhibitors include netarsudil. Examples of NO donor agents include latanoprostene bunod.

In one embodiment, the ocular drug delivery system of the invention comprises one or more anti-glaucoma drugs selected from latanoprost, bimatoprost, travoprost, tafluprost, latanoprostene bunod, pilocarpine, echothiophate, carbachol, timolol, nadolol, carteolol, levobunolol, metipranolol, betaxolol, brimonidine, apraclonidine, dorzolamide, brinzolamide, acetalozamide, methazolamide, and netarsudil.

In one embodiment, the ocular drug delivery system of the invention comprises one or more anti-glaucoma drugs. In one embodiment, the ocular drug delivery system of the invention comprises one anti-glaucoma drug. In one embodiment, the ocular drug delivery system of the invention comprises two or more anti-glaucoma drugs. In one embodiment, a combination of two or more IOP lowering agents is used, preferably a combination of drugs belonging to at least two different classes of IOP lowering agents.

In one embodiment, the ocular drug delivery system of the invention comprises at least one prostaglandin analog, preferably selected from latanoprost, bimatoprost, travoprost, tafluprost, latanoprostene bunod and combinations thereof. In a specific embodiment, the ocular delivery system of the invention comprises bimatoprost.

In one embodiment, the ocular drug delivery system of the invention comprises at least one beta blocker; preferably selected from timolol, nadolol, carteolol, levobunolol, metipranolol, betaxolol and combinations thereof. In a specific embodiment, the ocular delivery system of the invention comprises timolol.

In one embodiment, the ocular drug delivery system of the invention comprises at least one prostaglandin analog and at least one beta blocker. In one embodiment, the ocular drug delivery system of the invention comprises at least one prostaglandin analog selected from latanoprost, bimatoprost, travoprost, tafluprost and, latanoprostene bunod, and at least one beta blocker selected from timolol, nadolol, carteolol, levobunolol, metipranolol and betaxolol. In one embodiment, the ocular drug delivery system of the invention comprises bimatoprost and timolol.

In one embodiment, the ocular drug delivery system of the invention comprises at least one carbonic anhydrase inhibitor; preferably selected from dorzolamide, brinzolamide, acetazolamide, methazolamide and combinations thereof.

In one embodiment, the ocular drug delivery system of the invention comprises at least one prostaglandin analog and at least one carbonic anhydrase inhibitor. In one embodiment, the ocular drug delivery system of the invention comprises at least one prostaglandin analog selected from latanoprost, bimatoprost, travoprost, tafluprost and, latanoprostene bunod, and at least one carbonic anhydrase inhibitor selected from dorzolamide, brinzolamide, acetazolamide, and methazolamide.

In one embodiment, the ocular drug delivery system of the invention comprises at least one alpha adrenergic agonist; preferably selected from brimonidine, apraclonidine and combinations thereof.

In one embodiment, the ocular drug delivery system of the invention comprises at least one prostaglandin analog and at least one alpha adrenergic agonist. In one embodiment, the ocular drug delivery system of the invention comprises at least one prostaglandin analog selected from latanoprost, bimatoprost, travoprost, tafluprost and, latanoprostene bunod, and at least one alpha adrenergic agonist selected from brimonidine, and apraclonidine.

According to one embodiment, the ocular delivery system of the invention comprises one or more anti-glaucoma drug(s) in an amount ranging from 0.01% to 50% in weight of the total weight of the delivery system; preferably from 0.1% w/w to 20% w/w; more preferably from 0.1% w/w to 10% w/w; more preferably from 0.1% w/w to 5% w/w.

In one embodiment, the ocular delivery system of the invention comprises one or more anti-glaucoma drug and at least one supplementary pharmaceutically active substance. The supplementary pharmaceutically active substance may be for example selected from anti-inflammatories and dry eye treatment agents. Preferably, the supplementary pharmaceutically active substance is an ophthalmic drug.

Examples of anti-inflammatories comprise corticosteroids anti-inflammatory drugs (dexamethasone, fluorometholone, rimexolone, fluocinolone, fluticasone, loteprednol) and nonsteroidal anti-inflammatory drugs (bromfenac sesquihydrate, amfenac, nepafenac, aspirin, ibuprofen, ketorolac, tromethamine, diclofenac, flurbiprofen). Examples of dry eye treatment agents include immunosuppressive agents such as ciclosporin or tacrolimus.

According to a further embodiment, the ocular delivery system of the invention also enables to deliver other active substances including alleviating agents of ocular conditions such as dry eye. Examples of such alleviating agents include lubricating agents such as polyvinyl acid (PVA) or polyvinylpyrrolidone (PVP, also referred to as povidone).

### Preactivated Thiomers of Hyaluronic Acid

The ocular delivery system of the invention comprises at least one preactivated thiomer of hyaluronic acid. The presence of a preactivated thiomer of hyaluronic acid in the delivery system of the invention confers mucoadhesive properties to the system and enables to achieve expected delivery properties, preferably a release rate of more than 2 days, and more preferably of more than 3 days. Moreover, the presence of the preactivated thiomer of hyaluronic acid in the insert triggers swelling upon hydration and the formation of a gel. The use of hyaluronic acid as backbone of the preactivated thiomer is advantageous in terms of ophthalmic tolerance, hydrating and lubricating properties, and gelation properties. Hyaluronic acid is known to be well adapted to ocular use.

In the ocular delivery system of the invention, the preactivated thiomer of hyaluronic acid forms a matrix enabling to form an insert or a film with mucoadhesive properties. This matrix also enables to carry the anti-glaucoma drugs and to deliver them at the eye level.

Hyaluronic acid (HA) is a linear polysaccharide, which basic structure consists of repeating disaccharide units, namely D-glucuronic acid and N-acetyl glucosamine linked via β (1,4) and β (1,3) glucosidic bonds:

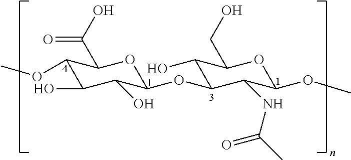

Preactivated thiomers are polymeric compounds bearing side chains containing vitamin B derivatives-disulfide groups, said side chains being covalently bonded to the polymeric backbone. Therefore, a “preactivated thiomer of hyaluronic acid” refers to hyaluronic acid modified by the presence of side chains comprising vitamin B derivatives-disulfide groups, preferably side chains comprising groups selected from (iso)nicotinic acid-disulfide, (iso) nicotinamide-disulfide and mercaptopyridoxines-disulfide groups, especially 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups.

It is herein defined that a group “2-nicotininic acid-disulfide” refers to the following group:

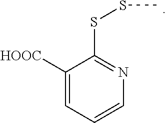

The groups 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups are defined accordingly.

The preactivated thiomers of hyaluronic acid can be obtained by two routes of synthesis: (a) a two-step synthesis or (b) a one-step synthesis.

In the two-step synthesis (a), the hyaluronic acid backbone is first modified by the covalent bonding of ligands containing free thiol groups (step a1), leading to a thiomer of hyaluronic acid. In the second step (step a2), the free thiol groups of the previously introduced side chains are preactivated by the formation of disulfide bonds with vitamin B derivatives, leading to the preactivated thiomer of hyaluronic acid.

By “thiomer of hyaluronic acid” or “thiolated hyaluronic acid” it is referred to hyaluronic acid onto which backbone are covalently bonded ligands containing free thiol groups as side chains. Two chemical groups of HA, namely carboxylic acid and hydroxyl groups, can be modified to form thiolated HA via different reactions, such as for example by amidation on the carboxylic acid or by ether or ester formation on hydroxyl groups. Examples of ligands that can be used to form thiolated HA by amidation of the carboxylic acid include cysteine, homocysteine, N-acetylcysteine, cysteine ethyl ester, cysteamine, mercaptoaniline, adipic acid dihydrazide (ADH)thiolated by reaction with Traut's reagent (iminothiolane), 5,5′-dithiobis(2-nitrobenzoic acid), dithiobis(propanoic dihydrazide), dithiobis(butyric dihydrazide), 3-(2-pyridyldithio) propionyl hydrazide, and dithiothreitol. Examples of ligands that can be used to form thiolated HA by ether formation on hydroxyl groups include ethylene sulfide. Examples of ligands that can be used to form thiolated HA by ester formation on hydroxyl groups include thioglycolic acid, 3-thiopropionic acid, 4-thiobutanoic acid, mercaptobenzoic acid, mercaptonicotinic acid, glutathione, and gamma-thiobutyrolactone.

By way of example of the two-step synthesis (a), the scheme below illustrates the thiolation of carboxylic acid groups of HA by amidation with an amino-thiol ligand (which can be for example cysteine or cysteamine) to form thiolated HA (step a1) and subsequently the protection of free thiol groups via preactivation using 6-mercaptonicotinamide:

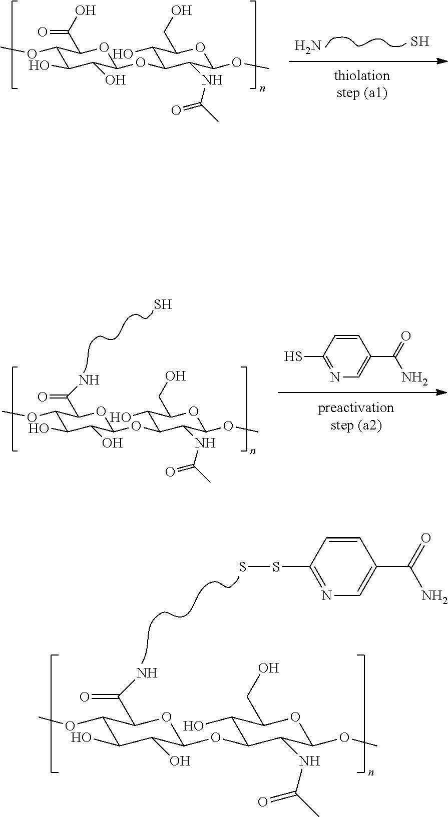

The synthesis of preactivated thiomers of hyaluronic acid can thus be performed by reaction of a thiolated hyaluronic acid backbone with 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, 6,6′-dithionicotinamide or 6-mercaptopyridoxine. In one embodiment, the preactivated thiomers of hyaluronic acid used in the ocular delivery system of the invention are manufactured according to the methods disclosed in US 2012/0225024.

In one embodiment, the preactivated thiomer of hyaluronic acid is selected from polymeric compounds bearing 2-mercaptonicotinic acid, 6-mercaptonicotinic acid, 2-mercaptonicotinamide, 2-mercaptoisonicotinamide, 6-mercaptonicotinamide, 6-mercaptoisonicotinamide, or 6-mercaptopyridoxine side chains covalently bonded through disulfide bonds to a thiolated hyaluronic acid backbone.

In the alternative route of synthesis, the one-step synthesis (b), the hyaluronic acid backbone is directly modified by the covalent bonding of ligands containing vitamin B derivatives-disulfide groups, leading to the preactivated thiomer of hyaluronic acid.

By way of example of the one-step synthesis (b), the scheme below illustrates the modification of carboxylic acid groups of HA by amidation with a (6-nicotinic acid)-disulfide-amino ligand (for example S-(6-mercaptonicotinic acid)-cysteine-disulfide or S-(6-nicotinic acid)-cysteamine-disulfide) to form the corresponding preactivated thiomer of HA:

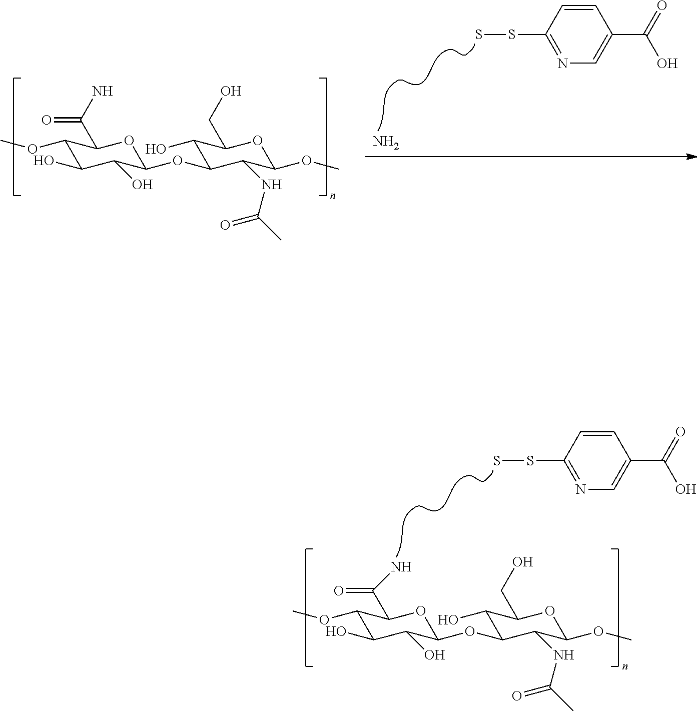

The synthesis of preactivated thiomers of hyaluronic acid can thus be performed by reaction of hyaluronic acid with ligands containing vitamin B derivatives-disulfide groups. Such disulfide ligands can be disulfide adducts made from:
one vitamin B derivative, such as 2-mercaptonicotinic acid (2-MNA), 6-mercaptonicotinic acid (6-MNA), 2-mercaptonicotinamide (2-MNAmide), 2-mercaptoisonicotinamide, 6-mercaptonicotinamide (6-MNAmide), 6-mercaptoisonicotinamide or 6-mercaptopyridoxine; and
one thiolated ligand such as cysteine, homocysteine, N-acetylcysteine, cysteine ethyl ester, cysteamine, mercaptoaniline, adipic acid dihydrazide (ADH)thiolated by reaction with Traut's reagent (iminothiolane), 5,5′-dithiobis(2-nitrobenzoic acid), dithiobis(propanoic dihydrazide), dithiobis(butyric dihydrazide), 3-(2-pyridyldithio) propionyl hydrazide, dithiothreitol, ethylene sulfide, thioglycolic acid, 3-thiopropionic acid, 4-thiobutanoic acid, mercaptobenzoic acid, mercaptonicotinic acid, and glutathione.

In one embodiment, the disulfide ligand used in the one-step synthesis (b) to form a preactivated thiomer of hyaluronic acid is selected from:
2-((2-aminoethyl)disulfaneyl)nicotinic acid (i.e. 2-MNA/cysteamine);
6-((2-aminoethyl)disulfaneyl)nicotinic acid (i.e. 6-MNA/cysteamine);
2-((2-amino-2-carboxyethyl)disulfaneyl)nicotinic acid (i.e. 2-MNA/cysteine);
6-((2-amino-2-carboxyethyl)disulfaneyl)nicotinic acid (i.e. 6-MNA/cysteine);
2-((2-aminoethyl)disulfaneyl) nicotinamide (i.e. 2-MNAmide/cysteamine);
6-((2-aminoethyl)disulfaneyl) nicotinamide (i.e. 6-MNAmide/cysteamine);
S-((3-carbamoylpyridin-2-yl)thio) cysteine (i.e. 2-MNAmide/cysteine); and
S-((5-carbamoylpyridin-2-yl)thio) cysteine (i.e. 6-MNAmide/cysteine).

Whatever the method of synthesis, the disulfide side chains of the preactivated thiomer of hyaluronic acid can be selected from:
S-(2- or 6-mercaptonicotinic acid)-, S-(2- or 6-mercapto (iso) nicotinamide)- or S-(6-mercaptopyridoxine)-cysteine-disulfides,
S-(2- or 6-mercaptonicotinic acid)-, S-(2- or 6-mercapto (iso) nicotinamide)- or S-(6-mercaptopyridoxine)-homocysteine-disulfides,
S-(2- or 6-mercaptonicotinic acid)-, S-(2- or 6-mercapto (iso) nicotinamide)- or S-(6-mercaptopyridoxine)-cysteamine-disulfides,
S-(2- or 6-mercaptonicotinic acid)-, S-(2- or 6-mercapto (iso) nicotinamide)- or S-(6-mercaptopyridoxine)-N-acetylcysteine-disulfides,
S-(2- or 6-mercaptonicotinic acid)-, S-(2- or 6-mercapto (iso) nicotinamide)- or S-(6-mercaptopyridoxine)-thioglycolic acid-disulfides,
S-(2- or 6-mercaptonicotinic acid)-, S-(2- or 6-mercapto (iso) nicotinamide)- or S-(6-mercaptopyridoxine)-3-thiopropionic acid-disulfides,
S-(2- or 6-mercaptonicotinic acid)-, S-(2- or 6-mercapto (iso) nicotinamide)- or S-(6-mercaptopyridoxine)-4-thiobutanoic acid-disulfides,
S-(2- or 6-mercaptonicotinic acid)-, S-(2- or 6-mercapto (iso) nicotinamide)- or S-(6-mercaptopyridoxine)-mercaptobenzoic acid-disulfides,
S-(2- or 6-mercaptonicotinic acid)-, S-(2- or 6-mercapto (iso) nicotinamide)- or S-(6-mercaptopyridoxine)-mercaptonicotinic acid-disulfides,
S-(2- or 6-mercaptonicotinic acid)-, S-(2- or 6-mercapto (iso) nicotinamide)- or S-(6-mercaptopyridoxine)-glutathione-disulfides, and
S-(2- or 6-mercaptonicotinic acid)-, S-(2- or 6-mercapto (iso) nicotinamide)- or S-(6-mercaptopyridoxine)-mercaptoaniline-disulfides;
   said side chains being independently attached to the hyaluronic acid backbone via amide, or ester bonds.

It is herein defined that S-(2- or 6-mercaptonicotinic acid)-stands for S-(2-mercaptonicotinic acid)- or S-(6-mercaptonicotinic acid)-; and that S-(2- or 6-mercapto (iso) nicotinamide)-stands for S-(2-mercaptonicotinamide)-, S-(2-mercaptoisonicotinamide)-, S-(6-mercaptonicotinamide)- or S-(6-mercaptoisonicotinamide)-.

The mucoadhesive properties of the preactivated thiomer can be modulated by adjusting the functionalization degree of the hyaluronic acid backbone by the vitamin B derivative-disulfide side chains. Further, it was evidenced that increasing the functionalization degree may advantageously enable to reduce the amount of preactivated thiomer to be used in the delivery system, while maintaining the mucoadhesive properties. In one embodiment, the preactivated thiomer of hyaluronic acid comprises from 10 μmol to 1350 μmol of mercaptonicotinic acid, mercaptonicotinamide, mercaptoisonicotinamide or mercaptopyridoxine partial structures per gram polymer; preferably from 10 μmol to 1000 μmol per gram polymer, preferably from 50 μmol to 500 μmol per gram polymer; more preferably from 50 μmol to 200 μmol per gram polymer. In one embodiment, the preactivated thiomer of hyaluronic acid comprises from μmol to 1350 μmol of mercaptonicotinic acid, mercaptonicotinamide, mercaptoisonicotinamide or mercaptopyridoxine partial structures per gram polymer; preferably from 100 μmol to 1350 μmol per gram polymer, preferably from 150 μmol to 1350 μmol per gram polymer; more preferably from 200 μmol to 1350 μmol per gram polymer.

The viscosity of the preactivated thiomer can be modulated by varying the molecular weight of the polymer. The molecular weight of the hyaluronic acid backbone also influences its functionalization degree. Especially, it was observed that the functionalization degree tends to decrease when the molecular weight increases, thereby influencing the mucoadhesion. Further, the molecular weight of the polymer influences the swelling of the delivery system of the invention: the swelling increases with molecular weight. Preferably, preactivated thiomers of hyaluronic acid with medium molecular weight are used, preferably with molecular weight ranging from 100 kDa to 1200 kDa, preferably from 100 kDa to 1000 kDa, more preferably from 200 kDa to 800 kDa. In one embodiment, the preactivated thiomers of hyaluronic acid has a molecular weight ranging from 100 kDa to 1200 kDa, preferably from 200 kDa to 1200 kDa.

In one embodiment, the preactivated thiomer of hyaluronic acid does not comprise free thiol groups. The absence of free thiol groups can be achieved by manufacturing the preactivated thiomer either by the one-step route of synthesis (b) described above; or by using the two-step method (a), in which step (a2) is conducted in conditions such that all the free thiols of the thiolated backbone are preactivated.

In another embodiment, the preactivated thiomer of hyaluronic acid comprises preactivated disulfide side chains and free thiol side chains. Free thiol side chains can be present when the preactivated thiomer of hyaluronic acid is obtained by the two-step method (a) described above, with first thiolation of the HA backbone, followed by preactivation of a fraction of the free thiol groups. The free thiol side chains are those forming the thiolated hyaluronic acid backbone before preactivation. Free thiol side chains can be selected from cysteine, homocysteine, N-acetylcysteine, cysteine ethyl ester, cysteamine, mercaptoaniline, adipic acid dihydrazide (ADH)thiolated by reaction with Traut's reagent (iminothiolane), 5,5′-dithiobis(2-nitrobenzoic acid), dithiobis(propanoic dihydrazide), dithiobis(butyric dihydrazide), 3-(2-pyridyldithio) propionyl hydrazide, dithiothreitol, ethylene sulfide, thioglycolic acid, 3-thiopropionic acid, 4-thiobutanoic acid, mercaptobenzoic acid, mercaptonicotinic acid, glutathione, and gamma-thiobutyrolactone. The amount of remaining free thiol side chains can be controlled by varying the amount of added vitamin B derivative during step (a2).

The presence of free thiol groups in the preactivated thiomer of hyaluronic acid may influence the gelation rate of the ocular system, since the presence of free thiol groups accelerated the formation of a gel upon hydration. The presence of free thiol groups may also lead to the crosslinking of the thiomer matrix. The crosslinking of the thiomer matrix can be modulated by the ratio of free thiol groups which in turns can modulate the cohesivity of the gel formed upon hydration.

In one embodiment, the preactivated thiomer of hyaluronic acid is partially crosslinked through the formation of disulfide bonds. Crosslinking may occur when the preactivated thiomer of hyaluronic acid comprises free thiol groups. In such case, crosslinking may occur (i) within one single polymer chain, between the different free thiol groups present on the side chains; (ii) between two chains of preactivated thiomer comprising free thiol groups; or (iii) between a preactivated thiomer of hyaluronic acid comprising free thiol groups and another species present in the ocular system that comprises free thiol groups. Case (iii) may occur for example when the ocular system of the invention also comprises a non-preactivated thiomer, as detailed hereafter. Alternatively, crosslinking may occur when the preactivated thiomer of hyaluronic acid does not comprise any free thiol group, but when the ocular system of the invention also comprises a species comprising free thiol groups, such as a non-preactivated thiomer, which may trigger S-deprotection in the preactivated thiomer.

The crosslinking degree of the thiomer matrix of the ocular system of the invention influences the cohesiveness of the gel formed upon hydration. Cohesiveness is an important property of the ocular system of the invention since it impacts the physical integrity of the gel over time which in turns affects the drug release profile. Indeed, the more the system is cohesive, the longer it remains on the eye, thereby prolonging the drug release profile. Cohesiveness can be evaluated by rheology measurements (such as storage and loss moduli, dynamic viscosity) and by assessing the swelling of the system during water uptake following hydration.

In one embodiment, the preactivated thiomers of hyaluronic acid present in the ocular delivery system of the invention are under the form of nanofibers; preferably nanofibers obtained by electrospinning. This presents the advantage to enable to control the rate of release of the anti-glaucoma drugs present in the delivery system by varying the density of the network of nanofibers.

According to one embodiment, the ocular delivery system of the invention comprises at least one preactivated thiomer of hyaluronic acid in an amount ranging from 5% to 99.99% in weight of the total weight of the delivery system (w/w), preferably from 10% to 99.99% w/w. In another embodiment, the ocular delivery system of the invention comprises at least one preactivated thiomer of hyaluronic acid in an amount ranging from 5% to 99.99% in weight of the total weight of the delivery system (w/w), preferably from 5% to 80% w/w, more preferably from 5% to 50% w/w, more preferably from 5% to 30% w/w, more preferably from 5% to 25% w/w.

According to one embodiment, the ocular delivery system of the invention comprises, further to the at least one preactivated thiomer of hyaluronic acid, one or more other preactivated thiomer wherein the polymeric backbone is other than hyaluronic acid.

### Non-Preactivated Thiomers

In one embodiment, the ocular delivery system of the invention further comprises a non-preactivated thiomer, i.e. a thiomer.

In a preferred embodiment, the non-preactivated thiomer is a thiomer of hyaluronic acid.

According to one embodiment, the thiolation of the hyaluronic acid backbone can be performed by coupling-via amide, ether, or ester bonds-moieties selected from cysteine, homocysteine, N-acetylcysteine, cysteine ethyl ester, cysteamine, mercaptoaniline, adipic acid dihydrazide (ADH)thiolated by reaction with Traut's reagent (iminothiolane), 5,5′-dithiobis(2-nitrobenzoic acid), dithiobis(propanoic dihydrazide), dithiobis(butyric dihydrazide), 3-(2-pyridyldithio) propionyl hydrazide, dithiothreitol, ethylene sulfide, thioglycolic acid, 3-thiopropionic acid, 4-thiobutanoic acid, mercaptobenzoic acid, mercaptonicotinic acid, glutathione, and gamma-thiobutyrolactone. Preferably, the thiolation of the polymeric backbone is performed by coupling cysteine or cysteamine.

In one embodiment, the non-preactivated thiomer, preferably the thiomer of hyaluronic acid, comprises from 100 μmol to 1500 μmol of thiol groups per gram polymer; preferably from 100 μmol to 800 μmol per gram polymer; preferably from 200 μmol to 800 μmol per gram polymer.

In one embodiment, the non-preactivated thiomers present in the ocular delivery system of the invention are under the form of nanofibers; preferably nanofibers obtained by electrospinning.

According to one embodiment, the ocular delivery system of the invention comprises a non-preactivated thiomer, preferably a thiomer of hyaluronic acid, in an amount ranging from 0% to 89.99% in weight of the total weight of the delivery system (w/w); preferably from 0% to 30% w/w; more preferably from 0% to 25% w/w; more preferably from 0% to 20% w/w.

In one embodiment, the presence of non-preactivated thiomers in the ocular system of the invention advantageously leads to the crosslinking of the thiomer matrix, and thereby increases the cohesiveness of the system and the drug release profile.

### Excipients

In one embodiment, the ocular delivery system of the invention comprises excipients, preferably pharmaceutically acceptable excipients. Such suitable excipients will be clear to the skilled person; reference is made to the latest edition of Remington's Pharmaceutical Sciences.

According to one embodiment, the ocular delivery system of the invention comprises one or more pharmaceutically acceptable excipient selected from: thickening agents, gelling agents, plasticizers, solubilization agents, stabilizing agents, permeation enhancers, diluents, binding agents, glidants, channeling agents, lubricants and modified release agents.

Examples of thickening and gelling agents include high molecular weight crosslinked polyacrylic acid polymers (e.g. Carbopol), polyvinyl alcohol, polyvinylpyrrolidone (PVP, also referred to as povidone), cellulose derivatives (e.g. hydroxypropyl methylcellulose (HPMC; also referred to as hypromellose), carboxymethylcellulose (CMC), hydroxypropylcellulose (HPC)), polyethylene glycol (PEG), and hyaluronic acid.

Examples of plasticizers include glycerol and polyethylene glycol (PEG).

Examples of solubilization and stabilizing agents, especially for active pharmaceutical ingredients, include cyclodextrins and non-ionic surfactants.

Examples of permeation enhancers include glutathione in its reduced form (GSH; 0.1%-1% w/w).

Examples of diluents include sugar alcohols such as sorbitol, trehalose, xylitol or mannitol.

Examples of binding agents include saccharides and their derivatives; disaccharides such as sucrose or lactose; polysaccharides and their derivatives such as starches, cellulose or modified cellulose such as microcrystalline cellulose and cellulose ethers such as hydroxypropyl cellulose (HPC); sugar alcohols such as xylitol, sorbitol or mannitol; synthetic polymers such as polyvinylpyrrolidone (PVP, also referred to as povidone) or polyethylene glycol (PEG).

Examples of glidants include magnesium stearate, dibasic calcium phosphate, starch, microcrystalline cellulose and colloidal silicon dioxide.

Examples of channeling agents include sodium chloride (NaCl) and polyethylene glycol (PEG) of molecular mass of 400 to 1500 g/mol.

Examples of lubricants include soluble lubricants, such as polyethylene glycol (PEG), polyoxyethylene stearates, lauryl sulphate salts, hydrogenated coco monoglycerides, hydrogenated coco monoglycerides diglycerides and hydrogenated coco monoglycerides triglycerides (Hard Fat-Witepsol®); and insoluble lubricants, such as magnesium stearate, glyceryl behenate, stearic acid, and glyceryl palmitostearate.

Examples of modified release agents include glyceryl dibehenate, glyceryl distearate, ammonio methacrylate copolymer (Type A), polyvinyl acetate-povidone co-polymer (Kollidon® SR), and polyvinyl caprolactam-polyvinyl acetate-polyethylene glycol graft co-polymer (Soluplus®).

According to one embodiment, the ocular delivery system of the invention comprises one or more pharmaceutically acceptable excipient selected from high molecular weight crosslinked polyacrylic acid polymers, polyvinyl alcohol, polyvinylpyrrolidone (also referred to as povidone), cellulose, microcrystalline cellulose, hydroxypropyl cellulose, hydroxypropyl methylcellulose (also referred to as hypromellose), carboxymethyl cellulose, polyethylene glycol, hyaluronic acid, glycerol, cyclodextrins, glutathione in its reduced form, sorbitol, trehalose, xylitol, mannitol, saccharides and their derivatives, sucrose, lactose, polysaccharides and their derivatives, magnesium stearate, dibasic calcium phosphate, colloidal silicon dioxide, sodium chloride, polyoxyethylene stearates, lauryl sulphate salts, hydrogenated coco monoglycerides, hydrogenated coco monoglycerides diglycerides and hydrogenated coco monoglycerides triglycerides, glyceryl behenate, stearic acid, glyceryl palmitostearate, glyceryl dibehenate, glyceryl distearate, ammonio methacrylate copolymer (Type A), polyvinyl acetate-povidone co-polymer, and polyvinyl caprolactam-polyvinyl acetate-polyethylene glycol graft co-polymer.

According to one embodiment, the ocular delivery system of the invention comprises one or more pharmaceutically acceptable excipient selected from polyvinylpyrrolidone (also referred to as povidone), hydroxypropyl methylcellulose (also referred to as hypromellose), magnesium stearate, glyceryl dibehenate, and mixtures thereof. According to one embodiment, the ocular delivery system of the invention comprises one or more pharmaceutically acceptable excipient selected from polyvinylpyrrolidone (also referred to as povidone), hydroxypropyl methylcellulose (also referred to as hypromellose), magnesium stearate, and mixtures thereof.

According to one embodiment, the ocular delivery system of the invention comprises one or more pharmaceutically acceptable excipient in an amount ranging from 0% to 94.99% in weight of the total weight of the delivery system (w/w), preferably from 0% to 94.5% w/w; preferably from 0% to 89.99% w/w. In one embodiment, the amount of pharmaceutically acceptable excipient(s) ranges from 40% to 94.5% w/w.

The excipients present in the ocular delivery system of the invention may enable to control its swelling upon hydration. Indeed, it should be avoided that the system gains too much volume upon hydration, otherwise it will no more be compatible with ocular use. In the case of an excessive swelling upon hydration, the ocular delivery system from the invention may be expulsed from the conjunctival cul-de-sac: the natural movements of the eyeball combined together with the permanent blinking of the eyelids creates important shear movements on the ocular delivery system. That would in turn significantly reduce its time of residence on the ocular surface.

The excipients present in the ocular delivery system of the invention may also enable to control the hardness of the ocular delivery system, especially in the case of systems used under dry form.

The choice of the excipients present in the ocular delivery system of the invention may also enable to control the rate of release of the active substance present in the delivery system.

### Ocular Delivery Systems' Compositions

Therefore, the ocular delivery system of the invention comprises:
one or more anti-glaucoma drug; and
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups.

Therefore, the ocular delivery system of the invention comprises:
one or more anti-glaucoma drug; and
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups; and wherein the preactivated thiomer of hyaluronic acid has a molecular weight ranging from 100 kDa to 1200 kDa.

In one embodiment, the ocular delivery system of the invention comprises:
one or more anti-glaucoma drug;
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups;
optionally a non-preactivated thiomer; and
optionally one or more pharmaceutically acceptable excipient.

In one embodiment, the ocular delivery system of the invention comprises:
one or more anti-glaucoma drug;
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups; and wherein the preactivated thiomer of hyaluronic acid has a molecular weight ranging from 100 kDa to 1200 kDa;
optionally a non-preactivated thiomer; and
optionally one or more pharmaceutically acceptable excipient.

In one embodiment, the ocular delivery system of the invention comprises:
0.01% to 50% in weight of the total weight of the delivery system (w/w) of one or more anti-glaucoma drug; preferably from 0.1% w/w to 20% w/w; more preferably from 0.1% w/w to 10% w/w; and
5% w/w to 99.99% w/w of at least one preactivated thiomer of hyaluronic acid as herein defined.

In one embodiment, the ocular delivery system of the invention comprises:
0.01% to 50% in weight of the total weight of the delivery system (w/w) of one or more anti-glaucoma drug; preferably from 0.1% w/w to 20% w/w; more preferably from 0.1% w/w to 10% w/w; and
10% w/w to 99.99% w/w of at least one preactivated thiomer of hyaluronic acid as herein defined.

In one embodiment, the ocular delivery system of the invention comprises:
0.01% to 50% in weight of the total weight of the delivery system (w/w) of one or more anti-glaucoma drug; preferably from 0.1% w/w to 20% w/w; more preferably from 0.1% w/w to 10% w/w; and
5% w/w to 80% w/w of at least one preactivated thiomer of hyaluronic acid as herein defined.

In one embodiment, the ocular delivery system of the invention comprises:
0.01% to 50% in weight of the total weight of the delivery system (w/w) of one or more anti-glaucoma drug; preferably from 0.1% w/w to 20% w/w; more preferably from 0.1% w/w to 10% w/w;
10% w/w to 99.99% w/w of at least one preactivated thiomer of hyaluronic acid as herein defined;
0% w/w to 89.99% w/w of a non-preactivated thiomer; and
0% w/w to 89.99% w/w of one or more pharmaceutically acceptable excipient.

In one embodiment, the ocular delivery system of the invention comprises:
0.01% to 50% in weight of the total weight of the delivery system (w/w) of one or more anti-glaucoma drug; preferably from 0.1% w/w to 20% w/w; more preferably from 0.1% w/w to 10% w/w;
5% w/w to 80% w/w of at least one preactivated thiomer of hyaluronic acid as herein defined;
0% w/w to 89.99% w/w of a non-preactivated thiomer; and
0% w/w to 94.99% w/w of one or more pharmaceutically acceptable excipient.

In one embodiment, the ocular delivery system of the invention comprises:
0.1% to 10% in weight of the total weight of the delivery system (w/w) of one or more anti-glaucoma drug; preferably from 0.1% w/w to 5% w/w;
5% w/w to 25% w/w of at least one preactivated thiomer of hyaluronic acid as herein defined;
0% w/w to 25% w/w of a non-preactivated thiomer; and
0% w/w to 94.5% w/w of one or more pharmaceutically acceptable excipient; preferably 40% w/w to 94.5% w/w.

In one embodiment, the ocular delivery system of the invention comprises:
0.1% to 10% in weight of the total weight of the delivery system (w/w) of one or more anti-glaucoma drug; preferably from 0.1% w/w to 5% w/w;
5% w/w to 25% w/w of at least one preactivated thiomer of hyaluronic acid as herein defined, wherein the preactivated thiomer of hyaluronic acid has a molecular weight ranging from 100 kDa to 1200 kDa;
0% w/w to 25% w/w of a non-preactivated thiomer; and
0% w/w to 94.5% w/w of one or more pharmaceutically acceptable excipient; preferably 40% w/w to 94.5% w/w.

In one embodiment, the ocular delivery system of the invention comprises:
0.1% to 10% in weight of the total weight of the delivery system (w/w) of one or more anti-glaucoma drug; preferably from 0.1% w/w to 5% w/w;
5% w/w to 25% w/w of at least one preactivated thiomer of hyaluronic acid as herein defined, wherein the preactivated thiomer of hyaluronic acid has a molecular weight ranging from 100 kDa to 1200 kDa; preferably from 200 kDa to 1200 kDa; and comprises from 100 μmol to 1350 μmol of mercaptonicotinic acid, mercaptonicotinamide, mercaptoisonicotinamide or mercaptopyridoxine partial structures per gram polymer; preferably from 150 μmol to 1350 μmol per gram polymer; preferably from 200 μmol to 1350 μmol per gram polymer;
0% w/w to 25% w/w of a non-preactivated thiomer; and
0% w/w to 94.5% w/w of one or more pharmaceutically acceptable excipient; preferably 40% w/w to 94.5% w/w.

### Properties of the Ocular Delivery System

The ocular delivery system of the invention presents mucoadhesive properties, especially ocular mucoadhesive properties, i.e. it strongly interacts with mucus or mucosal membranes, especially mucus or mucosal membranes of the eye. The mucoadhesive properties of the ocular delivery system of the invention can be measured by rotating cylinder method, as described in the art. The mucoadhesive properties of the ocular delivery system of the invention enable to achieve at least 2 days of mucoadhesion, preferably at least 3 days, more preferably from 3 to 8 days of mucoadhesion, even more preferably from 3 to 10 days of mucoadhesion. The mucoadhesive properties may be modulated by varying the functionalization degree of the hyaluronic acid backbone by the vitamin B derivative-disulfide side chains in the preactivated thiomer of hyaluronic acid used in the system.

The ocular delivery system of the invention enables to efficiently release the one or more anti-glaucoma drug present therein to the subject to which it is administered. Advantageously, the ocular delivery system of the invention enables a sustained release of the one or more anti-glaucoma drug to the eye. The release of the one or more anti-glaucoma drug can be assessed by dissolution testing, e.g. by placing the delivery system in water (or in a simulated lacrimal fluid) and by sampling the solution at different time points to measure the amount of drug released therein. In one embodiment, the one or more anti-glaucoma drug is released in the eye in therapeutically effective amount for at least 2 days, at least 3 days, at least 4 days, at least 5 days, at least 6 days, at least 7 days.

The release profile is influenced by the cohesiveness of the ocular system once hydrated. The more the system is cohesive, the longer it remains on the eye, thereby prolonging the drug release profile. Cohesiveness can be modulated by the crosslinking degree of the thiomer matrix of the system, i.e. the matrix made of preactivated thiomer of hyaluronic acid, and when present of non-preactivated thiomer.

After being placed in the conjunctival cul-de-sac or at the conjunctival surface, the ocular delivery system of the invention either completely dissolves during its period of use, or it needs to be removed after a given period of time (for example after several days). In case it needs to be removed, an eye wash solution can be used therefor.

The ocular delivery system of the invention can be used under dry or hydrated form. In one embodiment, the ocular delivery system of the invention is used under hydrated form, i.e. the preactivated thiomer matrix is hydrated under the form of a hydrogel before administration. In another embodiment, the ocular delivery system of the invention is under dry form, i.e. it contains a limited, if any, amount of water. In such case, once placed into the conjunctival cul-de-sac or at the conjunctival surface, the delivery system of the invention hydrates in situ and the hydrated preactivated thiomer matrix forms a mucoadhesive hydrogel (i.e. in situ gelation process).

In one embodiment, the hydration rate of the ocular delivery system of the invention is ranging from 1 minute to 24 hours, preferably from 1 minute to 1 hour; more preferably from 1 minute to 30 minutes.

Upon hydration, the ocular delivery system of the invention undergoes swelling. The swelling ratio of the ocular delivery system of the invention needs to be controlled, especially in case of direct use under dry form. The swelling ratio can be measured gravimetrically: (1) the system is weighted (W₀); (2) then it is immersed in water, or in a solvent such as simulated lacrimal fluid; (3) at several time points, the system is taken out, excess water is removed by gentle soaking with a tissue paper and weighted again (Wₜ). The amount of water uptake (i.e. swelling ratio, in weight %) is calculated as [(Wₜ−W₀)/W₀]*100. In one embodiment, the maximum swelling ratio of the ocular delivery system of the invention once hydrated is ranging from 0% to 10000% in weight to the weight of the dry form (w/w); preferably from 0% to 5000% w/w; more preferably from 0% to 2000%. In one embodiment, the maximum swelling ratio of the ocular delivery system of the invention once hydrated is ranging from 100% to 10000% in weight to the weight of the dry form (w/w); preferably from 100% to 5000% w/w; more preferably from 100% to 2500%, more preferably from 100% to 2000%, more preferably from 100% to 1500%. The swelling ratio depends on multiple factors including: the molecular weight of the preactivated thiomer, the amount of preactivated thiomer present in the system, the crosslinking degree of the matrix, the amount of non-preactivated thiomer present in the system. The size of the system and its hardness (especially when used under the form of an insert) also influence the swelling ratio.

The ocular delivery system of the invention can also be characterized by its “residence time” (or “time of residence”), i.e. the duration of the presence of the ocular delivery system at the eye level, especially in the lower conjunctival cul-de-sac. The residence time of the ocular delivery system of the invention can be measured by in vivo assay. The residence time of the ocular delivery system of the invention is preferably of at least 2 days, more preferably at least 3 days, even more preferably from 3 to 10 days. The residence time depends on multiple factors including the size and shape of the system, its mucoadhesiveness, its cohesivity, its erosion, and its swelling behavior. These parameters can be influenced by the composition of the delivery system.

The solid or semisolid ocular delivery system of the invention may be an ocular insert or an ocular film.

In one embodiment, the solid or semisolid ocular delivery system of the invention is an ocular insert, i.e. a solid or semisolid consistency tridimensional device designed to be placed into the conjunctival cul-de-sac or at the conjunctival surface, whose size and shape are especially designed for ophthalmic application. In one embodiment, the ocular insert is under dry form. In another embodiment, the ocular insert is hydrated and is under the form of a hydrogel pellet.

In one embodiment, the ocular delivery system of the invention is an electrospun ocular insert. Preferably the ocular insert is solely formed from the matrix of preactivated thiomer of hyaluronic acid, the one or more anti-glaucoma drug, optionally comprising a non-preactivated thiomer and excipient, but does not comprise any additional layers or other materials.

Ocular inserts can be obtained by direct compression of a mixture comprising the one or more anti-glaucoma drug dispersed in the preactivated thiomer of hyaluronic acid, which can be under lyophilized form.

The ocular insert can be of any shape and size, provided that it is suitable for ocular placement, and preferably is in shape of a rod, strip, thread, doughnut, disc, oval or quarter moon. In one embodiment, the ocular insert has one convex side and one concave side. The ocular insert does not display any angle on its surface and presents a smooth surface that does not cause irritation to the eye nor to the eyelids. Preferably, the cross section of the ocular insert is circular, square or rectangular. Preferably, the insert is sized and shaped to readily fit into the eye, or a part thereof. In one embodiment, the ocular insert has a thickness ranging from 0.1 mm to 5 mm, preferably from 0.5 mm to 2 mm, more preferably from 0.5 mm to 1.5 mm. In one embodiment, the ocular insert has a length ranging from 1 mm to 10 mm, preferably from 2 mm to 5 mm. In one embodiment, the ocular insert has a width ranging from 1 mm to 10 mm, preferably from 2 mm to 5 mm.

In another embodiment, the solid or semisolid ocular delivery system of the invention is an ocular film, i.e. a solid or semisolid consistency bidimensional film designed to be placed into the conjunctival cul-de-sac or at the conjunctival surface, whose size and shape are especially designed for ophthalmic application. In one embodiment, the ocular film is under dry form. In another embodiment, the ocular film is under hydrated form.

The ocular film can be square shaped, circular, ellipsoid or any other suitable shape. Preferably, the ocular film has a thickness ranging from 0.01 μm to 1000 μm, preferably 0.5 μm to 500 μm. In case the ocular film is a circular film, it may have a diameter ranging from 2 mm to 20 mm, preferably from 5 mm to 10 mm. The ocular film can also have a curvature for suitable placement on the surface of the eye.

Ocular films can be obtained by solvent evaporation of a solution comprising the preactivated thiomer of hyaluronic acid and one or more anti-glaucoma drug. Alternatively, ocular films can also be obtained by printing technologies, such as for example inkjet printing.

In one embodiment, the mucoadhesive solid or semisolid ocular delivery system of the invention is in unit dosage form.

The present invention further relates to a kit comprising the solid or semisolid ocular delivery system of the invention. The kit may comprise instructions for use in the treatment of glaucoma. The kit may also comprise an applicator, preferably a sterile applicator. The kit may also comprise an eye wash solution in case the delivery system needs to be removed from the eye.

### Glaucoma Treatment

The present invention also relates to the use of the mucoadhesive solid or semisolid ocular delivery system of the invention in the treatment of glaucoma. Especially, the ocular delivery system of the invention is useful to deliver one or more anti-glaucoma drug at the eye level of a subject. The targeted eye tissue can be, but is not limited to, corneal tissue, conjunctiva, eyelids, trabeculum, iris, ciliary body, uvea, choroid, retina or macula. The ocular delivery system of the invention is useful for human and veterinary uses.

In one embodiment, the invention provides a mucoadhesive solid or semisolid ocular delivery system of the invention for use in the treatment of glaucoma.

The invention also relates to the use of a mucoadhesive solid or semisolid ocular delivery system of the invention for the manufacturing of a medicament for the treatment of glaucoma.

The invention further relates to a method for treating glaucoma in a subject, comprising placing a mucoadhesive solid or semisolid ocular delivery system according to the invention in the conjunctival cul-de-sac or at the conjunctival surface of the subject in need thereof. The mucoadhesive solid or semisolid ocular delivery system of the invention is preferably placed in the cul-de-sac of the eye of the subject in need thereof.

In one embodiment, the glaucoma is open angle glaucoma.

In one embodiment, the use of the ocular delivery system of the invention enables to decrease the intraocular pressure in the eye of the subject.

### BRIEF DESCRIPTION OF THE DRAWINGS

FIG. 1 is a graph showing the dose (%) of bimatoprost released from the insert of formula F1 overtime in an in vitro release assay, calculated as concentration of drug released to theoretical concentration for 100% of drug released.

FIG. 2 is a graph showing the dose (%) of bimatoprost released from insert of formula F2 overtime in an in vitro release assay.

FIG. 3 is a graph showing the dose (%) of bimatoprost and timolol released from insert of formula F3 overtime in an in vitro release assay.

### EXAMPLES

The present invention is further illustrated by the following examples.

### Example 1: Ocular Inserts Manufacturing

Ocular inserts according to the invention with various formulations (F1-F4) were manufactured.

| | F1 | F2 | F3 | F4 |
|---|---|---|---|---|
| | (% | (% | (% | (% |
| Components | w/w) | w/w) | w/w) | w/w) |
| bimatoprost | 1 | 1 | 1 | |
| timolol | | | 17.5 | |
| latanoprost | | | | 0.18 |
| preactivated thiomer of | 99 | 25 | 11 | |
| HA (125 μmol/g; 0.5 MDa) | | | | |
| preactivated thiomer of | | | | 25 |
| HA (100 μmol/g; 0.8 MDa) | | | | |
| non-preactivated thiomer of | | 25 | 8.5 | 25 |
| HA (200 μmol/g; 0.8 MDa) | | | | |
| glyceryl dibehenate | | | | 48 |
| hypromellose | | 47 | 60 | |
| magnesium stearate | | 2 | 2 | 1.82 |
| Total | 100 | 100 | 100 | 100 |

Two different preactivated thiomers of HA were used. They differed by their molecular weight and by the functionalization degree of the HA backbone by the disulfide side chains, but bear the same side chains, namely 2-((2-aminoethyl)disulfaneyl)nicotinic acid (2-AMENA, corresponding to 2-MNA/cysteamine disulfide). These preactivated thiomers of HA were obtained by adapting the method described in US2012/0225024, using hyaluronic acid as polymeric backbone, cysteamine as ligand comprising free thiol groups, and 2-mercaptonicotinic acid (2-MNA) as preactivating group.

The non-preactivated thiomer of HA bore cysteamine side chains and had a molecular weight of 0.8 MDa and a functionalization degree of 200 μmol/g. The non-preactivated thiomers of HA was obtained by adapting the method described in US2012/0225024, using hyaluronic acid as polymeric backbone and cysteamine as ligand comprising free thiol groups.

The inserts were manufactured by mixing the components which are all under the form of powders. For formulations F1 and F4, the powder mixtures were first granulated by compaction followed by milling before being compressed. Inserts with formulations F2 and F3 were obtained by direct compression. Inserts of 10 mg, were prepared (length 4.3 mm, width 2.3 mm, thickness 1.1 mm).

### Example 2: In Vitro Mucoadhesion Assay

Purpose: This in vitro assay aims at determining the mucoadhesive properties of the inserts of the invention by the rotating cylinder method. This method consists in a visual test to evaluate the retention capability of the insert on mucosa while being subjected to shear.

Method: The inserts of example 1 were deposited on fresh animal mucosa. The mucosal surface was placed in a mesh disk (diameter 5 cm). A vessel of dissolution test apparatus (apparatus USP 2-method USP 5-Paddle-over-disk apparatus) was filled with simulated lacrimal fluid (sodium bicarbonate, 0.2% w/w; calcium chloride dihydrate, 0.008% w/w; sodium chloride, 0.67% w/w; ultrapure water, qsp; pH adjusted to 7.4 with HCl 5M) and the cylinder was rotated with 50 rev/min speed of rotation at a temperature of 32° C. The detachment or disintegration of the insert was visually determined.

Results: The duration of the retention of the inserts on the mucosa is reported in the table below:

| Insert | F1 | F2 | F3 | F4 |
|---|---|---|---|---|
| mucoadhesion (days) | 2 | 4 | 6 | NA |

Conclusion: The inserts of the invention enabled to achieve at least 2 days of mucoadhesion, and even 6 days with formulation F3.

### Example 3: In Vitro Swelling Assay

Purpose: This in vitro assay aims at determining the swelling behavior of the inserts of the invention by measuring their ability to uptake water.

Method: Water uptake was determined gravimetrically: (1) Inserts of example 1 were weighted (W₀); (2) then they were immersed in a simulated lacrimal fluid (cf example 2), in a closed container to prevent evaporation during the assay and incubated at 32° C., the eye surface temperature; (3) at several time points, the system is taken out, excess water is removed by gentle soaking with a tissue paper and weighted again (Wₜ). The amount of water uptake (i.e. swelling ratio, in weight %) is calculated as [(Wₜ−W₀)/W₀]*100.

Results: The swelling ratio of the inserts is reported in the table below:

| Insert | F1 | F2 | F3 | F4 |
|---|---|---|---|---|
| swelling ratio (% w/w) | 9500 | 4800 | 1300 | NA |
| time to reach the swelling ratio (h) | 24 | 6 | 2 | NA |

Conclusion: The swelling behavior of the inserts of the invention can be modulated by varying their composition.

### Example 4: In Vivo Residence Time Evaluation

Purpose: This in vivo evaluation aims at determining the time of residence of the inserts of the invention by measuring the duration of the presence of the insert in the lower conjunctival cul-de-sac of rabbits, as a result of its mucoadhesion properties, hydration and swelling behavior and formulation characteristics.

### Methods

Ocular inserts F5-F7, whose composition is detailed below, were manufactured as reported in Example 1. The preactivated thiomer of HA used in F5-F7 has the same ide chains being 2-AMENA as in Example 1, but with a different molecular weight (0.6 MDa) and a different functionalization degree (110 μmol/g). The non-preactivated thiomer of HA is the same as in Example 1.

The swelling ratio was measured in vitro as in Example 3.

The residence time was measured as follows. An insert was assayed unilaterally in the inferior conjunctival cul-de-sac of New Zealand white rabbit's eye (n=3) by pulling the lower eyelid, then the insert was placed with tweezers. The lid was let back to its normal place. The presence of the insert in the lower conjunctival cul-de-sac was monitored twice a day (morning and afternoon) until the absence of the insert was observed. The time of residence of the insert in the lower conjunctival cul-de-sac was then recorded.

### Results

| Inserts' Components (% w/w) | F5 | F6 | F7 |
|---|---|---|---|
| bimatoprost | 1 | 1 | 1 |
| preactivated thiomer of HA | 25 | 20 | 10 |
| (110 μmol/g; 0.6 MDa) | | | |
| non-preactivated thiomer of HA | 25 | | 10 |
| (200 μmol/g; 0.8 MDa) | | | |
| povidone | 15 | 14 | 14 |
| hypromellose | 32 | 63 | 63 |
| magnesium stearate | 2 | 2 | 2 |
| Total | 100 | 100 | 100 |
| Swelling ratio (% w/w) | 2500 | 1200 | 950 |
| Residence Time (days) | 2 | 7 | 9 |

Conclusion: The time of residence of the inserts of the invention on the ocular surface can be modulated by varying their composition.

### Example 5: Drug Release Profile

Purpose: This assay aims at determining the drug release profile overtime.

Method: Inserts of example 1 were placed in glass flasks filed with 200 mL purified water. The flasks were placed in an oscillating water bath at 32° C. Samples (10 mL) were taken at predetermined time points and 10 mL of purified water were added to maintain the 200 mL total volume. In each sample, the concentration of released anti-glaucoma drug was measured by HPLC-UV. The result is expressed in dose percent calculated as the concentration of drug released to the theoretical concentration for 100% of drug released.

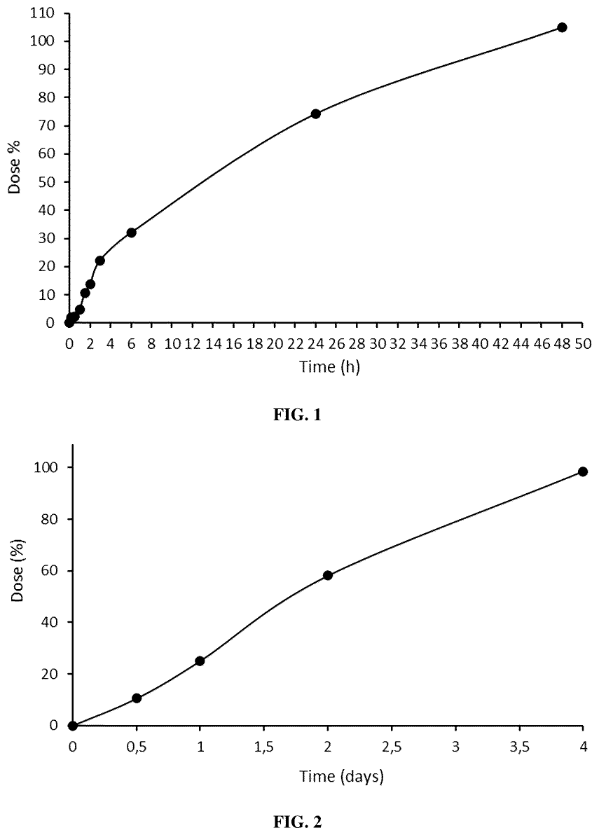

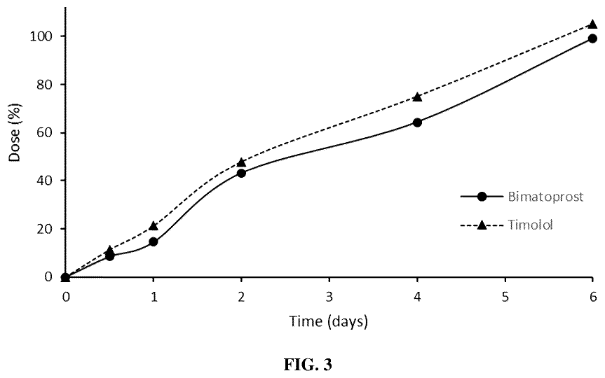

Results: The amount of anti-glaucoma drug released from the tested insert overtime is reported in FIGS. 1, 2 and 3 for inserts of formulation F1, F2 and F3 respectively.

Conclusion: The inserts of the invention enabled to obtain a controlled release overtime of the anti-glaucoma drug, for at least 2 days. Formulation F3 even enabled to achieve a release over 6 days.

1-15. (canceled)

16. A mucoadhesive solid or semisolid ocular delivery system comprising:
one or more anti-glaucoma drug; and
at least one preactivated thiomer of hyaluronic acid selected from polymeric compounds having a hyaluronic acid backbone bearing covalently bonded side chains comprising groups selected from 2-nicotinic acid-disulfide, 6-nicotinic acid-disulfide, 2-nicotinamide-disulfide, 2-isonicotinamide-disulfide, 6-nicotinamide-disulfide, 6-isonicotinamide-disulfide, and 6-pyridoxine-disulfide groups; and
wherein the preactivated thiomer of hyaluronic acid has a molecular weight ranging from 100 kDa to 1200 kDa.

17. The ocular delivery system according to claim 16, which is an ocular insert or an ocular film.

18. The ocular delivery system according to claim 16, wherein the side chains of the preactivated thiomer of hyaluronic acid are selected from:
S-(2- or 6-mercaptonicotinic acid)-, S-(2- or 6-mercapto (iso) nicotinamide)- or S-(6-mercaptopyridoxine)-cysteine-disulfides,
S-(2- or 6-mercaptonicotinic acid)-, S-(2- or 6-mercapto (iso) nicotinamide)- or S-(6-mercaptopyridoxine)-homocysteine-disulfides,
S-(2- or 6-mercaptonicotinic acid)-, S-(2- or 6-mercapto (iso) nicotinamide)- or S-(6-mercaptopyridoxine)-cysteamine-disulfides,
S-(2- or 6-mercaptonicotinic acid)-, S-(2- or 6-mercapto (iso) nicotinamide)- or S-(6-mercaptopyridoxine)-N-acetylcysteine-disulfides,
S-(2- or 6-mercaptonicotinic acid)-, S-(2- or 6-mercapto (iso) nicotinamide)- or S-(6-mercaptopyridoxine)-thioglycolic acid-disulfides,
S-(2- or 6-mercaptonicotinic acid)-, S-(2- or 6-mercapto (iso) nicotinamide)- or S-(6-mercaptopyridoxine)-3-thiopropionic acid-disulfides,
S-(2- or 6-mercaptonicotinic acid)-, S-(2- or 6-mercapto (iso) nicotinamide)- or S-(6-mercaptopyridoxine)-4-thiobutanoic acid-disulfides,
S-(2- or 6-mercaptonicotinic acid)-, S-(2- or 6-mercapto (iso) nicotinamide)- or S-(6-mercaptopyridoxine)-mercaptobenzoic acid-disulfides,
S-(2- or 6-mercaptonicotinic acid)-, S-(2- or 6-mercapto (iso) nicotinamide)- or S-(6-mercaptopyridoxine)-mercaptonicotinic acid-disulfides,
S-(2- or 6-mercaptonicotinic acid)-, S-(2- or 6-mercapto (iso) nicotinamide)- or S-(6-mercaptopyridoxine)-glutathione-disulfides, and
S-(2- or 6-mercaptonicotinic acid)-, S-(2- or 6-mercapto (iso) nicotinamide)- or S-(6-mercaptopyridoxine)-mercaptoaniline-disulfides;
said side chains being independently attached to the hyaluronic acid backbone via amide, or ester bonds.

19. The ocular delivery system according to claim 16, wherein the preactivated thiomer of hyaluronic acid comprises from 10 μmol to 1350 μmol of mercaptonicotinic acid, mercaptonicotinamide, mercaptoisonicotinamide or mercaptopyridoxine partial structures per gram polymer.

20. The ocular delivery system according to claim 16, wherein the preactivated thiomer of hyaluronic acid comprises from 100 μmol to 1350 μmol of mercaptonicotinic acid, mercaptonicotinamide, mercaptoisonicotinamide or mercaptopyridoxine partial structures per gram polymer.

21. The ocular delivery system according to claim 16, further comprising a non-preactivated thiomer.

22. The ocular delivery system according to claim 16, further comprising a non-preactivated thiomer of hyaluronic acid.

23. The ocular delivery system according to claim 21, wherein the non-preactivated thiomer is selected from polymeric compounds having a hyaluronic acid backbone bearing covalently bonded thiolated side chains selected from cysteine, homocysteine, N-acetylcysteine, cysteine ethyl ester, cysteamine, mercaptoaniline, adipic acid dihydrazide thiolated by reaction with iminothiolane, 5,5′-dithiobis(2-nitrobenzoic acid), dithiobis(propanoic dihydrazide), dithiobis(butyric dihydrazide), 3-(2-pyridyldithio) propionyl hydrazide, dithiothreitol, ethylene sulfide, thioglycolic acid, 3-thiopropionic acid, 4-thiobutanoic acid, mercaptobenzoic acid, mercaptonicotinic acid, glutathione, and gamma-thiobutyrolactone; said side chains being independently attached to the hyaluronic acid backbone via amide, ether or ester bonds.

24. The ocular delivery system according to claim 16, wherein the preactivated thiomer of hyaluronic acid, and/or when present the non-preactivated thiomer, is crosslinked.

25. The ocular delivery system according to claim 16, wherein the anti-glaucoma drug is an intraocular pressure (IOP) lowering agent selected from prostaglandin analogs, cholinomimetics, beta blockers, alpha adrenergic agonists, carbonic anhydrase inhibitors, Rho kinase inhibitors, NO donor agents and combinations thereof.

26. The ocular delivery system according to claim 16, wherein the anti-glaucoma drug is selected from latanoprost, bimatoprost, travoprost, tafluprost, latanoprostene bunod, pilocarpine, echothiophate, carbachol, timolol, nadolol, carteolol, levobunolol, metipranolol, betaxolol, brimonidine, apraclonidine, dorzolamide, brinzolamide, acetalozamide, methazolamide, and netarsudil.

27. The ocular delivery system according to claim 16, comprising two anti-glaucoma drugs, one being a prostaglandin analog and the other being a beta blocker.

28. The ocular delivery system according to claim 16, comprising two anti-glaucoma drugs, one being a prostaglandin analog which is bimatoprost and the other being a beta blocker which is timolol.

29. The ocular delivery system according to claim 16, further comprising one or more pharmaceutically acceptable excipient.

30. The ocular delivery system according to claim 29, wherein the excipient is selected from: thickening agents, gelling agents, plasticizers, solubilization agents, stabilizing agents, permeation enhancers, diluents, binding agents, glidants, channeling agents, lubricants and modified release agents.

31. The ocular delivery system according to claim 29, wherein the excipient is selected from: high molecular weight crosslinked polyacrylic acid polymers, polyvinyl alcohol, polyvinylpyrrolidone (also referred to as povidone), cellulose, microcrystalline cellulose, hydroxypropyl cellulose, hydroxypropyl methylcellulose (also referred to as hypromellose), carboxymethyl cellulose, polyethylene glycol, hyaluronic acid, glycerol, cyclodextrins, glutathione in its reduced form, sorbitol, trehalose, xylitol, mannitol, saccharides and their derivatives, sucrose, lactose, polysaccharides and their derivatives, magnesium stearate, dibasic calcium phosphate, colloidal silicon dioxide, sodium chloride, polyoxyethylene stearates, lauryl sulphate salts, hydrogenated coco monoglycerides, hydrogenated coco monoglycerides diglycerides and hydrogenated coco monoglycerides triglycerides, glyceryl behenate, stearic acid, glyceryl palmitostearate, glyceryl dibehenate, glyceryl distearate, ammonio methacrylate copolymer (Type A), polyvinyl acetate-povidone co-polymer, and polyvinyl caprolactam-polyvinyl acetate-polyethylene glycol graft co-polymer.

32. The ocular delivery system according to claim 16, comprising:
0.01% to 50% in weight of the total weight of the delivery system (w/w) of one or more anti-glaucoma drug;
5% to 80% w/w of at least one preactivated thiomer of hyaluronic acid;
0% to 89.99% w/w of a non-preactivated thiomer of hyaluronic acid; and
0% to 94.99% w/w of one or more pharmaceutically acceptable excipient.

33. A method for the treatment of glaucoma in a subject in need thereof comprising administering to said subject an ocular delivery system according to claim 16 in a therapeutically effective amount.
